(19) United States
(12) Patent Application Publication
(10) Pub. No.: US 2017/0188616 A1
(43) Pub. Date: Jul. 6, 2017

(54) CANNABINOID-ENRICHED SUPPLEMENT

(71) Applicant: THERABIS, LLC, Denver, CO (US)

(72) Inventors: Anthony James Alfiere, Scottsdale, AZ (US); Stephen M. Katz, Mohegan Lake, NY (US); James T. Sharkey, Peoria, AZ (US)

(21) Appl. No.: 15/382,653

(22) Filed: Dec. 17, 2016

Related U.S. Application Data
(60) Provisional application No. 62/268,984, filed on Dec. 17, 2015.

(51) Int. Cl. A23L 33/105; A23L 33/15; A23L 29/256; A23L 33/10; A61K 31/375; A61K 9/00; A61K 31/05; A61K 47/42; A61K 35/618; A23L 29/10; A23L 33/17

(52) U.S. Cl. CPC A23L 33/105; A23L 29/10; A23L 33/15; A23L 29/256; A23L 33/10; A23L 33/17; A61K 9/0056; A61K 31/05; A61K 47/42; A61K 35/618; A61K 31/375; A23V 2002/00

(57) ABSTRACT

A cannabinoid-enriched supplement includes a cannabinoid, an emulsifier, and a gelatin.

### CROSS REFERENCE TO RELATED APPLICATION

This application claims priority to and the benefit of U.S. Provisional Application No. 62/268,984, the contents of which are incorporated by reference.

### TECHNICAL FIELD

The disclosure is generally related to the field of delivery of cannabinoids to humans or animals. In particular, the disclosure pertains to the cannabinoid-enriched supplements.

### BACKGROUND

In some delivery mechanisms involving cannabinoids, other ingredients may reduce the delivery of the expected amount of cannabinoid to a human or animal. For example, fiber present in a delivery mechanism will bind with a portion of the cannabinoids, sometimes a large portion, rendering the full amount of cannabinoids unavailable for absorption by the human or animal. Other ingredients in a delivery mechanism may also inhibit the bioavailability of the cannabinoid. For example, agar, which will bind to cannabinoids, is used as a gelatin for stability of cannabinoid containing products.

Additionally, the delivery mechanism's production process can degrade or destroy the cannabinoids within the product. Cannabinoids can be especially sensitive to heat. For example, in a baking process for an animal supplement, the baking heat needed to create a product can also lead to thermal degradation of and loss of heat labile cannabinoids compounds. Such a process leads to more expense and waste of the effective ingredient, cannabinoids.

What is needed is a delivery mechanism creating greater bioavailability of cannabinoids. What is also needed is a production process that is sensitive to the cannabinoid's heat tolerance profile.

### SUMMARY

While the way in which the invention addresses the disadvantages of the prior art will be discussed in greater detail below, in general, the present invention provides for a cannabinoid-enriched supplement. An embodiment of cannabinoid-enriched supplement includes a cannabinoid, an emulsifier, and a gelatin formed into an edible product to be consumed by an animal or human. Another embodiment provides a consumable cannabinoid-enriched supplement comprising a first layer, a second layer disposed on top of the first layer, a third layer disposed on top of the second layer, a pocket enveloped in three dimensions by the second and third layers, the pocket having a cannabinoid mixed with an emulsifier.

Additional features and advantages of the invention will be set forth in the description which follows, and in part will be obvious from the description, or may be learned by practice of the invention. The features and advantages of the invention may be realized and obtained by means of the instruments and combinations particularly pointed out in the appended claims. These and other features of the present invention will become more fully apparent from the following description, or may be learned by the practice of the invention.

### BRIEF DESCRIPTION OF THE DRAWINGS

FIG. 1 shows a multi-layer cannabinoid-enriched supplement.

FIG. 2 shows a flowchart illustrating a method of making a cannabinoid-enriched supplement.

FIG. 3 shows a flowchart illustrating a method of making a liquid, cannabinoid-enriched supplement.

FIG. 4 shows a flowchart illustrating a method of making a cannabinoid-enriched supplement.

### DETAILED DESCRIPTION

Cannabis is being increasingly recognized for its potential use in medical applications humans and animals. For example, cannabis-enhanced products may be useful in treating neurological and inflammatory disorders or as a complement for longer-term treatments whose effects take relatively longer to be realized. Various embodiments of the invention are described in detail below. A person skilled in the relevant art will recognize that other components and configurations may be easily used or substituted than those that are described here without parting from the spirit and scope of the invention.

A cannabinoid-enriched supplement includes a cannabinoid, an emulsifier, and a gelatin formed into an edible product to be consumed by an animal or human. The cannabinoid may include any oil from any of the cannabis plant. In its various descriptions, a cannabinoid may refer to one cannabinoid or a mixture of cannabinoids, cannabinoid isolates, or any other isolated and purified cannabinoid, terpene, or biomolecule product by the genus Cannabis plant. The cannabinoid, in its various embodiments, is chosen based on the therapeutic effects desired in an application or delivery mechanism. For example, a common cannabinoid, called cannabidiol (CBD), is included in the supplement for its calming effect on the nervous system or alleviating pain. In other examples, other cannabinoids may be used to induce drowsiness or sleep, to suppress appetite, treat/inhibit tumors, reduce nausea or various other effects depending on the type of cannabinoid. Cannabinoids may be combined within a single supplement to provide multiple benefits at the same time. In general, the amount of cannabinoid content is 2-6 mg.

A gelatin is added to the cannabinoid to enhance the texture and thermal stability of the supplement. In some embodiments, the gelatin added is agar-agar, or simply known as agar. The use of a gelatin enables the supplement to take on multiple forms (e.g., different shapes and sizes) providing a pleasing texture to a human or animal and thus enhance its use. Although agar is a preferred gelatin, any gelatin that can achieve the same effects is suitable. Such gelatins are well known and will not be described in detail. The amount of gelatin is dependent on the desired composition of the supplement.

An emulsifier is added to the cannabinoid-gelatin mixture to enhance absorption of the cannabinoid by the human or animal. One issue with cannabinoids is that they will bind with various forms of fiber when combined with various ingredients. For example, the agar gelatin used to stabilize the supplement also lowers the bioavailability of the cannabinoid-gelatin mixture. Use of an emulsifier lowers the binding of cannabinoids with the fiber content of the agar. In some embodiments, the emulsifier is a polysorbate. The polysorbate class of emulsifiers have been show to enhance absorption of large molecules by the body. However, any emulsifier that aids absorption of a cannabinoid is suitable.

Other embodiments of a cannabinoid-enriched supplement include a cannabinoid-gelatin-emulsifier combination and an additive for enhancing the water-holding properties of a gel, or in other words, reducing syneresis. Syneresis is a condition where a gel structure is destabilized due to liquid separating from the gel. In its embodiments, the additive may include substance known as hypercolloids. Some example of such substances are pectin, carrageenan, and locust bean gum, however, any suitable substance that addresses the issue of syneresis in the supplement is suitable.

In some embodiments of a cannabinoid-enriched supplement, the supplement includes an anti-inflammatory compound. A preferred anti-inflammatory compound is green lipped mussel powder. The powder has been shown to address conditions involving joint pain. However, any anti-inflammatory compound is suitable for including in the supplement depending on its desired use. In some embodiments, the green lipped mussel powder is added to the cannabinoid at a ratio of 35 to 1. In alternative embodiments, the ratio is 30 to 1. However, any ratio suitable for end use is contemplated.

Many embodiments of a cannabinoid-enriched supplement will include a flavoring to enhance palatability. While any flavoring is contemplated, flavorings such as vegetable, beef, bacon, or chicken are preferred for animal directed embodiments.

Some embodiments of a cannabinoid-enriched supplement may contain a calming agent. For embodiments directed toward animal use, L-theanine is used to provide synergistic calming effects without overly sedating the animal. In some embodiments, wherein the cannabinoid is cannabidol, the L-theanine is added to the cannabinoid at a ratio of 15 to 1. In alternative embodiments, the ratio is 10 to 1. However, any ratio suitable for end use is contemplated. However, the amount added will be dependent on the desired effect in the intended user (e.g., type of animal, etc.)

Some embodiments of a cannabinoid-enriched supplement may contain an anti-allergy agent. For embodiments directed towards animals, a combination of bromelain and quercetin is used to product synergistic activity to suppress the allergic response in the animal. The combination mixed with the supplement produce additional synergistic effects due to the action on common biochemical pathways between the inflammatory and allergic response. In preferred embodiments, 100-200 mg of bromelain is added to the supplement. In preferred embodiments, 50-100 mg of quercetin is added to the supplement. In exemplary embodiments where both agents are used, the ratio of bromelain to quercetin is 2 to 1. In alternative embodiments, 200 mg of bromelain and 100 mg of quercetin is used. However, the amount added will be dependent on the desired effect in the intended user.

Additional ingredients may be added to the supplement depending on desired characteristics. For example, Valerian root (50-100 mg), chamomile (10-50 mg), peppermint (10-50 mg), turmeric (50-400 mg), dehydrated licorice root (100-200 mg) slippery elm (100-200 mg), and black cumin seeds (10-30 mg). Any ingredient or combination of these ingredients being added to a supplement is contemplated.

A cannabinoid-enriched supplement may be produced in many forms depending on the final use. For example, supplements directed towards animals, for example, dogs, may be produced as a dog treat or biscuit, kibble, or chew. The supplements may be single or multi-layer. In some embodiments directed towards animals, a cannabinoid-enriched supplement includes a cannabinoid mixture that includes 2-6 mg of CBD, 75-150 mg of green lipped mussel powder, 25-50 mg of vitamin C, and 125-250 mg of hemp powder. L-theanine may be added to the mixture, 25-50 mg, to enhance calming effects.

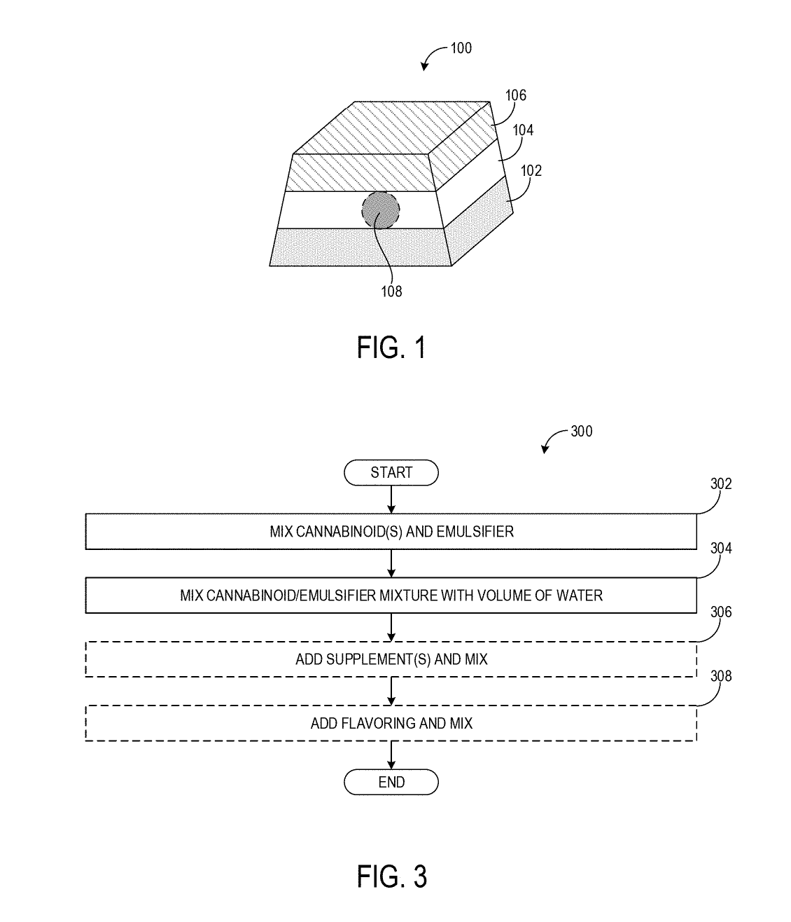

FIG. 1 shows an embodiment of the invention as a multi-layer cannabinoid-enriched supplement 100. In the depicted example, supplement 100 comprises three layers, though other layer numbers are contemplated as described in further detail below. The three layers include a bottom layer 102, an intermediate layer 104, and a top layer 106. Bottom layer 102 may comprise gelatin or another suitable substance, and may form the base of supplement 100. Placed vertically above bottom layer 102 is intermediate layer 104 which may also comprise gelatin or another suitable substance, and may at least partially surround in one, two, or three dimensions a pocket 108. As a non-limiting example, FIG. 1 shows intermediate layer 104 completely enveloping pocket 108, where the pocket is approximately centered in the intermediate layer in three dimensions. Pocket 108 includes a cannabinoid mixed with a suitable emulsifier. In exemplary embodiments, the pocket includes 5-15 mg of CBD mixed with a polysorbate emulsifier. By being at least partially surrounded by intermediate layer 104, and interposed between bottom and top layers 102 and 106, the cannabinoid is suspended in a fixed location and inhibited from inadvertent release such as by physically handling supplement 100. Further, the potential differing chemical composition of pocket 108 and the layers 102, 104, and 106 may allow a consumer to perceive and differentiate the portions of supplement 100 that include the cannabinoid and those that do not. In some examples, one or more of layers 102, 104, and 106 may be at least partially translucent, which may aid consumers in visually differentiating the portion of the supplement 100 that includes the cannabinoid.

Placed vertically above intermediate layer 104 and pocket 108 is top layer 106. Top layer 106 is shown in FIG. 1 as differing in appearance from those of layers 102 and 104 and pocket 108, as the top layer may differ in chemical composition from those of the other layers. For example, top layer 106 may comprise gelatin or another suitable substance mixed with a suitable flavoring agent—e.g., animal stock such as beef broth. Alternatively, or in addition, top layer 106 may include one or more substances for treating at least one health issue in a living being. As a non-limiting example, supplement 100 may be adapted for consumption by animals such as dogs, and may include a substance such as green lipped mussel extract for addressing joint inflammation. The inclusion of the cannabinoid in pocket 108 may complement the green lipped mussel extract or other suitable substance by enabling the realization of positive, tangible effects such as pain relief.

It will be appreciated that supplement 100 is provided as a non-limiting example, and that numerous aspects of the supplement may be varied without departing from the scope of this disclosure. For example, the ordering of layers 102, 104, and 106, and pocket 108, as well as their material composition, may be varied. In some examples, flavoring and/or medicinal substances such as green lipped mussel extract may be added to layers 102 and/or 104, alternatively or in addition to layer 106. Other substances may be added to one or more of layers 102, 104, and 106 alternatively or in addition to those mentioned above; such substances may include vitamin C, vitamin E, and/or chromium, for example.

Cannabinoid-enriched supplements comprising one or two layers are further contemplated. As a non-limiting example, a single layer cannabinoid-enriched supplement may include a single layer including gelatin and one or both of flavoring (e.g., animal stock) and green lipped mussel extract. The single layer product may be formed by disposing an initial layer of the gelatin mixture, disposing a pocket comprising a cannabinoid and an emulsifier on top of the initial layer, and enveloping the pocket with a layer of the gelatin mixture, for example. As another non-limiting example, a two-layer consumable cannabinoid-enriched supplement may include a first layer of gelatin or a gelatin mixture, a pocket comprising a metered dose of at least one cannabinoid and a suitable emulsifier on top of the first layer, and a second layer of gelatin or the gelatin mixture (e.g., gelatin, animal stock, and green lipped mussel extract) on top of and enveloping the pocket.

While described above with reference to CBD, it will be understood that pocket 108 may include additional or alternative cannabinoids. For example, in some implementations pocket 108 may include a combination of CBD and tetrahydrocannabinol (THC).

Other physical properties of product 100 may further be varied without departing from the scope of this disclosure. Such properties may include, for example, dimensions (e.g., length, height, width), shape, stiffness, appearance, etc.

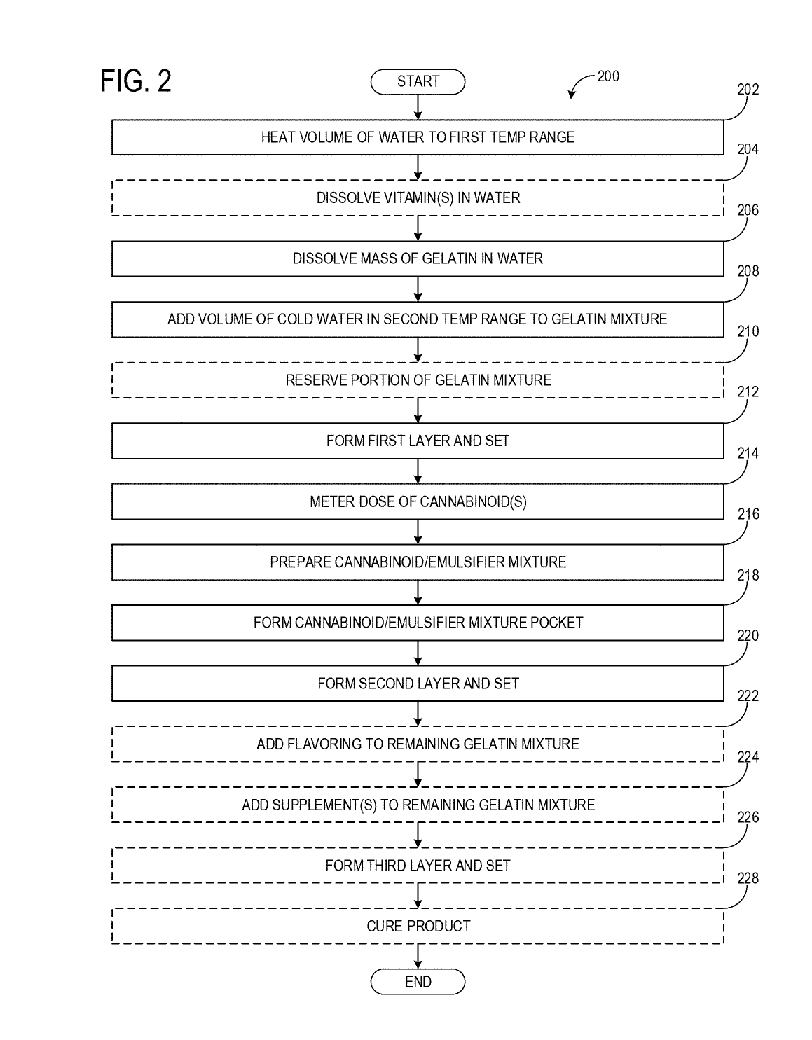

FIG. 2 shows a flowchart illustrating a method 200 of making a cannabinoid-enriched supplement. Method 200 may be employed to make supplement 100 of FIG. 1, for example.

At 202 of method 200, a volume of water is heated to a temperature in a first temperature range. For example, 4-5 mL of water may be heated to a temperature between 200 and 220° F.

At 204 of method 200, one or more vitamins may optionally be dissolved in the heated volume of water. Vitamin C and/or vitamin E (e.g., 350-450 IU) in crystal form, for example, may be dissolved in the heated water. As one non-limiting example, 300-400 mg of calcium ascorbate buffered or non-buffered vitamin C may be added to the heated water.

At 206 of method 200, a mass of gelatin is dissolved in the heated volume of water. For example, 1-2 g of gelatin may be dissolved in the heated water. Other suitable thickening agents may be alternatively used, however, including but not limited to pectin, xanthan gum, and agar.

At 208 of method 200, a volume of cold water in a second temperature range is added to the gelatin mixture (e.g., the mixture of water, gelatin or other thickening agent, and any vitamins optionally added at 204). For example, 4.5-5.5 mL of water in a temperature range of 35 to 45° F. may added to the gelatin mixture.

At 210 of method 200, a portion of the gelatin mixture may optionally be reserved. The optional reservation of the gelatin mixture may depend on the number of layers to be formed—for example, the portion of the gelatin mixture may not be reserved for single or two layer products.

At 212 of method 200, a first layer is formed and set. Setting the first layer may include refrigerating the first layer in a temperature range from 30 to 40° F. for between 15 and 30 minutes, for example. Forming the first layer may include disposing the first layer in a suitable mold, for example—e.g., a cubical mold having 1″ dimensions.

At 214 of method 200, a desired dose of one or more cannabinoids is metered. As a non-limiting example, a desired dose of 5-15 mg of CBD may be metered based on a known concentration of CBD oil comprising CBD.

At 216 of method 200, a cannabinoid/emulsifier mixture is prepared. The mixture may include at least one emulsifier and the metered dose of the at least one cannabinoid. As a non-limiting example, CBD and a suitable emulsifier may be prepared at a 1:1 ratio by weight, by mixing at a temperature between 100 to 120° F. until the CBD and emulsifier are blended to a desired degree. Suitable emulsifiers may include polysorbate 20/60/80 and coconut oil, for example.

At 218 of method 200, a pocket of the cannabinoid/emulsifier mixture prepared at 216 is formed. The pocket may be at least partially formed by disposing the cannabinoid/emulsifier mixture on top of the first layer. It will be appreciated that formation of a second layer on top of the pocket may complete formation of the pocket in some examples.

At 220 of method 200, the second layer is formed and set. Setting the second layer may include refrigerating the product in its instant form in a temperature range from 30 to 40° F. for between 15 and 30 minutes, for example. The second layer may be formed using the portion of the gelatin mixture optionally reserved at 210, for example, and may be disposed above the first layer and pocket.

At 222 of method 200, flavoring may optionally be added to the remaining gelatin mixture. As a non-limiting example, between 0.75 and 1.5 g of beef broth crystals may be added to the remaining gelatin mixture, though any suitable combination of flavorings may be used.

At 224 of method 200, one or more supplements may optionally be added to the remaining gelatin mixture. The one or more supplements may include, for example, 150-250 μg of chromium and/or between 200 and 300 mg of green lipped mussel extract may be added. As described above, the use of CBD and green lipped mussel extract may enable the product to address joint inflammation in animals in both the short and long term.

At 226 of method 200, a third layer is formed and set. Setting the third layer may include refrigerating the product in its instant form in a temperature range from 30 to 40° F. for between 15 and 30 minutes, for example. The third layer may be disposed above the first layer, pocket, and second layer. The third layer may be derived from the remaining gelatin mixture, for example.

At 228 of method 200, the product is optionally cured. Curing the product may include, for example, refrigerating the product in a temperature range from 30 to 40° F. overnight (or a comparable length of time).

Method 200 may be modified without departing from the scope of this disclosure. For example, method 200 may be modified to accommodate the formation of single or two layer products. Alternatively, or additionally, method 200 may be modified to modify the chemical composition of one or more of the first, second, and third layers, and the pocket—e.g., the first, second, and third layers may include a thickening agent, one or more supplements, and flavoring. For a two-layer product, for example, a portion of the gelatin mixture may be reserved prior to the addition of both flavoring and one or more supplements. Further, the amount of gelatin or other thickening agent may be adjusted to produce a product with a desired resistance to melting at room temperatures. Still further, it will be understood that the numerical ranges (e.g., temperature, volume, weight) provided above are exemplary and may be modified without departing from the scope of this disclosure.

Although supplement 100 (FIG. 1) may be approximately solid, the approaches described are applicable to the formation of cannabinoid-enriched supplements in non-solid form. FIG. 3 shows a flowchart illustrating a method 300 of making a liquid, cannabinoid-enriched supplement.

At 302 of method 300, a cannabinoid and an emulsifier are mixed. As a non-limiting example, CBD may be blended with an emulsifier at a 1:1 ratio by weight at 85-95° F. at 650-750 RPM.

At 304 of method 300, the cannabinoid/emulsifier mixture is mixed with a volume of water. As a non-limiting example, 0.75-1.25 mL of CBD/emulsifier mixture may be mixed with 13.5-14.5 mL of boiling water via agitation until a desired degree of uniformity is obtained.

At 306 of method 300, a supplement may optionally be added to the instant mixture and mixed. The supplement may include chromium (e.g., 150-250 μg) and/or green lipped mussel extract (e.g., between 200 and 300 mg, powder or liquid).

At 308 of method 300, flavoring is optionally added to the instant mixture and mixed. As a non-limiting example, beef bullion (e.g., 0.25-0.75 g) or beef broth may be added and mixed until dissolved at 80° F.

Following 308, the liquid mixture may be suitably packaged (e.g., bottled).

As a non-limiting example, a liquid, consumable cannabinoid-based product may include one or more of CBD oil (e.g., 5-15 mg), hemp oil (e.g., 1-2 mL), an emulsifier, green lipped mussel extract (e.g., 200-300 mg), chromium (e.g., 150-250 μg), vitamin C (e.g., 300-400 mg), vitamin E (e.g., 350-450 IU), water (e.g., a suitable volume to obtain a final liquid volume between 10 and 20 mL), and beef bullion (e.g., 0.25-1 g).

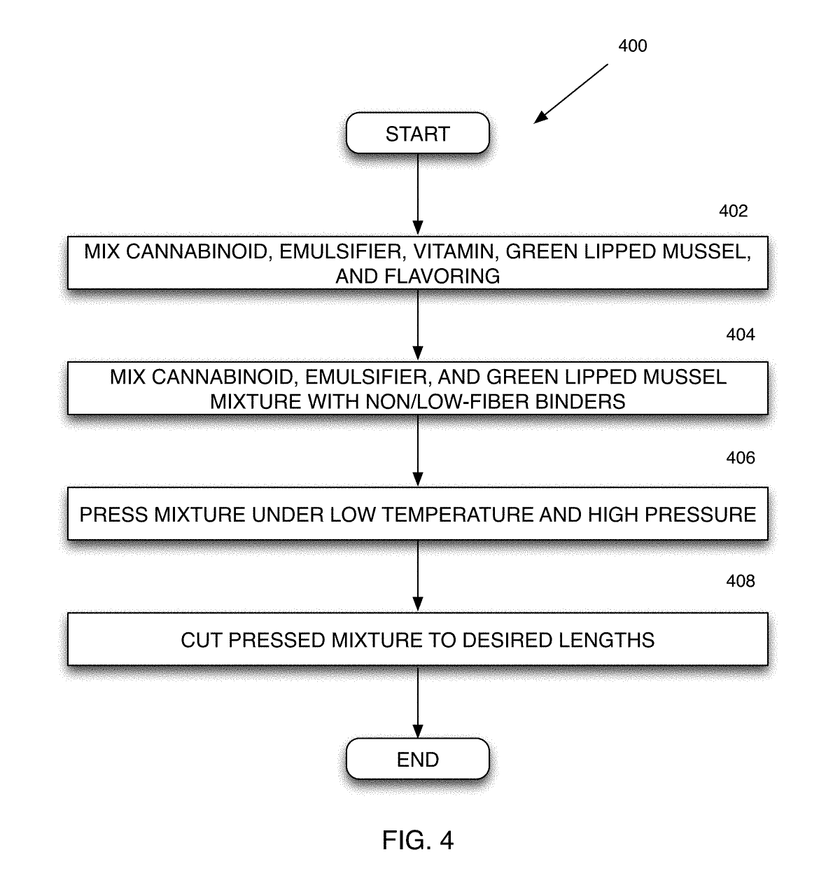

FIG. 4 shows a flowchart illustrating a method 400 of making a cannabinoid-enriched supplement by cold pressing. The cold pressed supplement may be single or multi-layered.

At 402 of method 400, a cannabinoid, an emulsifier, a vitamin, green lipped mussel powder, and a flavoring are mixed. As a non-limiting example, the supplement includes a cannabinoid that includes 2-6 mg of CBD, and 75-100 mg of green lipped mussel.

At 404 of method 400, non/low-fiber binders are added to the cannabinoid-emulsifier-mussel powder mixture. In a non-limiting example, the binders include honey, or coconut flour.

At 406 of method 400, the mixture is pressed or extruded at low temperature and high pressure. In a non-limiting example, the temperature may range from 40° F. to 100° F. and the pressure may range from 50,000 to 87,000 psi. The low temperature minimizes loss of the cannabinoids due to heating. However, any temperature and pressure to appropriately form the mixture is contemplated.

At 408 of method 400, the pressed/extruded mixture is cut at desired lengths to create a usable supplement. In a non-limiting example, the cut lengths are such that a supplement of 5-20 g is obtained.

Although the above description may contain specific details, they should not be construed as limiting the claims in any way. Other configurations of the described embodiments of the invention are part of the scope of this invention. The descriptions and embodiments are not intended to be an exhaustive or to limit the invention to the precise forms disclosed. Accordingly, the appended claims and their legal equivalents should only define the invention, rather than any specific examples given.

1. The cannabinoid-enriched supplement of claim 14, further comprising:
a first layer;
a second layer disposed on top of the first layer;
a third layer disposed on top of the second layer; and
a pocket enveloped in three dimensions by the second and third layers, the pocket having a metered dose of the cannabinoid mixed with the emulsifier.

2. The supplement of claim 1, wherein at least one of the first, second, and third layers include the first gelatin.

3. The supplement of claim 1, wherein at least one of the first, second, and third layers include a vitamin.

4. The supplement of claim 3, further comprising a green lipped mussel extract.

5. (canceled)

6. The supplement of claim 1, wherein at least one of the first, second, and third layers include a flavoring.

7. The supplement of claim 6, wherein the flavoring includes animal stock.

8. The supplement of claim 1, wherein the second layer is at least partially translucent so as to render the pocket at least partially visible.

9. The supplement of claim 1, wherein the cannabinoid includes cannabidiol.

10. The supplement of claim 1, wherein
the first layer includes the first gelatin and vitamin C,
the second layer includes a second gelatin,
the third layer includes a third gelatin, animal broth flavoring, and green lipped mussel extract, and
the cannabinoid is cannabidiol.

11. A method of making a cannabinoid-based product, comprising:
preparing a gelatin mixture;
forming and setting a first layer derived from the gelatin mixture;
preparing a cannabinoid-emulsifier mixture including a metered dose of at least one cannabinoid mixed with an emulsifier;
forming a pocket of the cannabinoid-emulsifier mixture on top of the first layer;
forming a second layer derived from the gelatin mixture on top of the pocket such that the pocket is enveloped in three dimensions; and
curing the product.

12. The method of claim 11, further comprising, before curing the product, forming a third layer derived from the gelatin mixture on top of the second layer,
wherein the gelatin mixture includes vitamin C,
wherein the third layer includes animal broth flavoring and green lipped mussel extract, and
wherein the at least one cannabinoid is cannabidiol.

13. A method of making a cannabinoid-enriched supplement comprising:
preparing a cannabinoid, emulsifier, and green lipped mussel powder mixture;
adding a binder to the mixture;
pressing the mixture under low heat and high pressure;
cutting the pressed mixture.

14. A cannabinoid-enriched supplement comprising:
a cannabinoid;
an emulsifier; and
a first gelatin.

15. The supplement of claim 14, wherein the cannabinoid is cannabidiol.

16. The supplement of claim 14, wherein the emulsifier is a polysorbate.

17. The supplement of claim 14, wherein the first gelatin is agar.

18. The supplement of claim 14, further comprising green lipped mussel powder.

19. The supplement of claim 14, further comprising at least one of L-theanine, bromelain, and quercetin.
